(19) United States
(12) Patent Application Publication
(10) Pub. No.: US 2024/0359007 A1
(43) Pub. Date: Oct. 31, 2024

(54) METHOD, SYSTEM AND ELECTRONIC WEARABLE UNIT FOR CONTROLLING ELECTRICAL STIMULATION TO PELVIC REGION OF A SUBJECT

(71) Applicant: INDIAN INSTITUTE OF SCIENCE, Kamataka, Bengaluru (IN)

(72) Inventors: Manish Arora, Bengaluru (IN); Komal Shah, Bengaluru (IN)

(21) Appl. No.: 18/287,843

(22) PCT Filed: Apr. 24, 2022
(86) PCT No.: PCT/IB2022/053798

§ 371 Date: Oct. 20, 2023

(30) Foreign Application Priority Data
Apr. 24, 2021 (IN) 202141012868

(51) Int. Cl. A61N 1/36; A61N 1/04

(52) U.S. Cl. CPC A61N 1/36031; A61N 1/0452; A61N 1/0456; A61N 1/0484; A61N 1/36007; A61N 1/36034

(57) ABSTRACT

Disclosed herein a method, tin Electrical Stimulation System (ESS) 101, and an electronic wearable unit 301 for controlling an electrical stimulation 113 to a pelvic region of a subject 103. The electronic wearable unit 301 comprises a non-skin contacting outer surface 307, a skin contacting inner surface 305 with a crotch part for removably attaching a urine collecting device 309, and the ESS 101 removably placed within a space bounded by the outer surface 307 and the inner surface 305. An input 105 for initiating a session is received. Further, at least one of, an activity level 107 of muscles in and around the pelvic region, an orientation 109 of one or more of the muscles, and a body posture 111 of the subject 103 is received, throughout the session. Based on the determination, the electrical stimulation 113 to the pelvic region of the subject 103 is controlled. Thus, the present disclosure enables portable administration of treatment, and further enables extending the electrical stimulation 113 from portable use in a fixed, body posture to ambulatory use, in multiple body postures.

### TECHNICAL FIELD

The present subject matter is generally related to electrical stimulation, and more particularly, but not exclusively, to a method, an Electrical Stimulation System (ESS) and an electronic wearable unit for controlling an electrical stimulation to a pelvic region of a subject.

### BACKGROUND

Pelvic floor muscles are located between a tailbone (coccyx) and a pubic bone within a pelvis of a subject. The pelvic floor muscles support a bladder, a rectum, a uterus (in female subject), and other abdominal organs against gravity and any added downward pressure. However, the pelvic floor muscles gradually weaken with natural aging process. Also, weakening of the pelvic floor muscles is caused due to pelvic surgery, childbirth (in female subject), long term coughing, constipation, repeated heavy lifting, and from being overweight. This results in dysfunction of the pelvic floor muscles in the subject.

Generally, pelvic floor muscle exercises are known as the first line of treatment for strengthening of pelvic floor muscles. However, about 50% subjects are not able to identify and isolate the pelvic floor muscles for correctly performing the exercises on their own. Further, the subject fails to assess regular progress associated with the exercise-based treatment. Moreover, recommended clinic visit schedule for the pelvic floor muscle exercises and/or physiotherapy based options is very taxing on a day-to-day basis. These result in lack of long-term compliance, and the subject desires for options beyond the pelvic floor muscle exercises for quick recovery of weak pelvic floor muscles.

Electrical stimulation is a widely accepted treatment solution for rehabilitation of the pelvic floor muscles in the subjects, especially for those who are suffering from urinary incontinence. In electrical stimulation, an electrical current is applied to the weak pelvic floor muscles to trigger contractive, and other types of movement and response in the pelvic floor muscles. Such a repetitive action strengthens the weak pelvic floor muscles, thereby making the electrical stimulation a potential substitute for the pelvic floor muscle exercises.

Generally, long-term electrical stimulation is recommended for subjects suffering from urinary incontinence. Either chronic or maximal stimulation may be provided based on the clinical requirements. While chronic stimulation is given at low intensity for several hours on daily basis, maximal stimulation uses maximum tolerable intensity currents for shorter periods. Both types typically require regular applications for several months.

Existing systems enable the subject to administer such stimulation-based treatment in a portable manner. However, for effective treatment, the subject needs to maintain a fixed posture throughout the stimulation session. In some scenarios, it becomes difficult for the subject to maintain the fixed posture for long hours, thus making it infeasible. This slows down the treatment progress and decreases treatment effectiveness and efficiency. Due to the aforesaid reasons, the subjects do not appreciate use of the existing systems for long hours on a day-to-day basis, and hence low compliance is common.

Further, the existing systems do not adapt the stimulation treatment to the change in the body posture, by any means such as modifying or adjusting electrical stimulation. As an example, when the subject is standing, a pelvic inlet is tilted anteriorly. But in upright position, bony arches of the pelvic inlet are oriented in an almost vertical plane. In the two cases, a gravitational force on a given muscle is different. Similarly, effect of gravity is eliminated when the subject is lying down or has a positive effect when the subject is sitting with a pillow under hips. The existing systems overlook such variation in the gravitational force exerted on the pelvic floor muscles with the change in the body posture of the subject, and thereby fail to provide appropriate electrical stimulation.

Moreover, in the existing systems, as described above, setting of various stimulation parameters is performed manually, which purely depends on expertise of the clinician or the physiotherapist and on the comfort level of the subject. This is highly subjective in nature and often reduces the effectiveness of the treatment. As the existing systems works in an open loop manner, accuracy in adjusting the stimulation parameters is not guaranteed for effective treatment.

Furthermore, existing systems do not facilitate management of the condition of urinary incontinence by the subject. As an example, when the subject visits workplace, the subject cannot use simultaneously, a urine collecting device such as an absorbent pad for holding urine, and one of the existing systems for treatment. Hence, existing systems do not provide flexibility and comfort to the subject and cannot reduce fear of social or workplace embarrassment associated with urinary incontinence.

The information disclosed in this background of the disclosure section is only for enhancement of understanding of the general background of the invention and should not be taken as an acknowledgement or any form of suggestion that this information forms the prior art already known to a person skilled in the art.

### SUMMARY

The present disclosure discloses a method of controlling an electrical stimulation to a pelvic region of a subject. The method comprises receiving, by an Electrical Stimulation System (ESS), one or more inputs for initiating a session. Upon receiving the one or more inputs, the method comprises receiving throughout the session, by the ESS, at least one of, an activity level of a plurality of muscles in and around the pelvic region, an orientation of one or more of the plurality of muscles, and a body posture of the subject. Thereafter, the method comprises controlling, by the ESS, the electrical stimulation to the pelvic region based on at least one of, the activity level of the plurality of muscles, the orientation of one or more of the plurality of muscles, and the body posture of the subject.

Further, the present disclosure discloses an Electrical Stimulation System (ESS) for controlling an electrical stimulation to a pelvic region of a subject. The ESS comprises a processor; and a memory communicatively coupled to the processor. The processor receives one or more inputs for initiating a session. Upon receiving the input, the processor receives throughout the session at least one of, an activity level of a plurality of muscles in and around the pelvic region, an orientation of one or more of the plurality of muscles, and a body posture of the subject. Based on at least one of, the activity level of the plurality of muscles, the orientation of one or more of the plurality of muscles, and the body posture of the subject, the processor controls the electrical stimulation to the pelvic region.

Furthermore, the present disclosure discloses an electronic wearable unit for controlling an electrical stimulation to a pelvic region of a subject. The electronic wearable comprises one or more non-electronic wearable parts and one or more electronic wearable parts. The one or more electronic wearable parts comprises at least a part of an Electrical Stimulation System (ESS). The ESS is configured to receive one or more inputs for initiating a session. Further, the ESS is configured to receive throughout the session at least one of, an activity level of a plurality of muscles in and around the pelvic region, an orientation of one or more of the plurality of muscles, and a body posture of the subject. Furthermore, the ESS is configured to control the electrical stimulation to the pelvic region based on at least one of, the activity level of the plurality of muscles, the orientation of one or more of the plurality of muscles, and the body posture of the subject.

Furthermore, the present disclosure discloses a method for rehabilitation of pelvic region of a subject by controlling an electrical stimulation to the pelvic region. The method comprises receiving, by an Electrical Stimulation System (ESS), one or more inputs for initiating a session. Upon receiving the one or more inputs, the method comprises receiving throughout the session, by the ESS, at least one of, an activity level of a plurality of muscles in and around the pelvic region, an orientation of one or more of the plurality of muscles, and a body posture of the subject. Thereafter, the method comprises controlling, by the ESS, the electrical stimulation to the pelvic region based on at least one of, the activity level of the plurality of muscles, the orientation of one or more of the plurality of muscles, and the body posture of the subject.

Furthermore, the present disclosure discloses a method for rehabilitation of pelvic region of a subject by controlling an electrical stimulation to the pelvic region. The method comprises receiving, by an Electrical Stimulation System (ESS), one or more inputs for initiating a session. Upon receiving the one or more inputs, the method comprises receiving throughout the session, by the ESS, at least one of, an activity level of a plurality of muscles in and around the pelvic region, an orientation of one or more of the plurality of muscles, and a body posture of the subject. Thereafter, the method comprises controlling, by the ESS, the electrical stimulation to the pelvic region based on at least one of, the activity level of the plurality of muscles, the orientation of one or more of the plurality of muscles, and the body posture of the subject.

Furthermore, the present disclosure discloses a method of managing disorder associated with pelvic region of a subject, said method comprising rehabilitation of the pelvic region by controlling an electrical stimulation to the pelvic region.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 shows an exemplary environment for controlling an electrical stimulation to a pelvic region of a subject in accordance with some embodiments of the present disclosure.

FIG. 2 shows a block diagram of an Electrical Stimulation System (ESS) in accordance with some embodiments of the present disclosure.

FIGS. 3a-3b show an electronic wearable unit for controlling an electrical stimulation to a pelvic region of a subject in accordance with some embodiments of the present disclosure.

FIGS. 3c-3d show placement of first electrodes and second electrodes adjacent to pelvic region of a subject in accordance with some embodiments of the present disclosure.

FIG. 4 shows a flow chart illustrating a method of controlling an electrical stimulation to a pelvic region of a subject in accordance with some embodiments of the present disclosure.

FIG. 5 shows a block diagram of an exemplary computer system for implementing embodiments consistent with the present disclosure.

It should be appreciated by those skilled in the art that any flow diagrams and timing diagrams herein represent conceptual views of illustrative device embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternatives falling within the scope of the disclosure.

The terms “comprises”, “comprising”, “includes”, “including” or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

Embodiments of the present disclosure relate to a method, an Electrical Stimulation System (ESS), and an electronic wearable unit for controlling an electrical stimulation to a pelvic region of a subject. In the present disclosure, the electrical stimulation is controlled based on at least one of, activity level of plurality of muscles in and around the pelvic region, orientation of one or more of the plurality of muscles, and body posture of the subject.

The present disclosure enables the subject to administer treatment in a portable manner, and further extends treatment from portable use in a fixed body posture to ambulatory use, in multiple body postures, while performing regular day-to-day activities. Due to automatic control, expertise and domain knowledge of the subject is not required for self-administration of the treatment. Further, electrical stimulation is automatically adjusted based on the real-time information about at least one of activity level of the plurality of muscles, orientation of one or more of the plurality of muscles and body posture of the subject. Such activity level of the plurality of muscles is determined from real-time Electromyography (EMG) signals associated with the plurality of muscles acquired by one or more EMG electrodes. In the present description, EMG signals received from the one or more EMG electrodes is considered as an example. However, this should not be considered as limiting. Signals based on pressure measurements received from instruments such as perineometer may be used to determine the activity level. A person skilled in the art will appreciate that the activity level may be measured by using other electrodes/instruments/devices and is not limited to above mentioned methods. Such orientation of one or more of the plurality of muscles and/or body posture of the subject is determined using orientation sensors. Thus, the present disclosure provides a closed loop control mechanism, in which the electrical stimulation is controlled based on real-time feedback. This ensures accuracy in adjusting stimulation parameters for effective treatment. Due to the real-time feedback, effect of gravity on the pelvic floor muscles is automatically considered while evaluating electrical current for providing the electrical stimulation.

Further, the ESS is operated in multiple operating modes such as a stimulation mode and an exercise mode. In the stimulation mode, the real-time feedback based on EMG readings acts as a response for the closed loop system for accurately controlling the electrical stimulation. Whereas in the exercise mode, the real-time feedback based on EMG readings acts as biofeedback for providing information to the subject regarding contraction of the plurality of muscles while the subject is performing exercises involving the plurality of muscles.

Further, the electronic wearable unit enables the subject to control the electrical stimulation and manage the condition of urinary incontinence simultaneously. In the electronic wearable unit, a skin contacting surface comprises a part in and around the crotch region of the subject, due to which the subject can attach and detach a urine collecting device such as an absorbent pad, without removing the ESS. In the present disclosure, the subject conceals the electronic wearable unit under regular clothing and administers the treatment in a discreet manner, utilizing a User Equipment (UE) which may be wirelessly coupled with the ESS. This enables the subject to flexibly control the electrical stimulation without requiring to be in a private venue or requiring access to the electronic wearable unit.

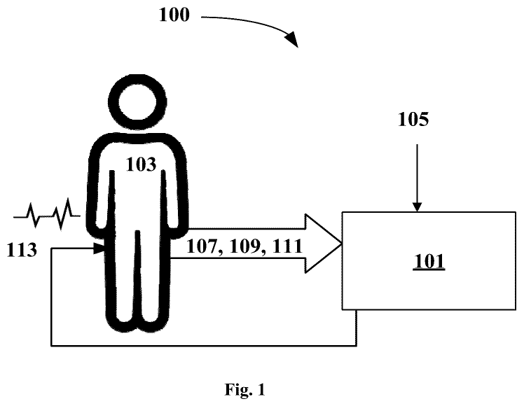

FIG. 1 shows an exemplary environment for controlling an electrical stimulation to a pelvic region of a subject in accordance with some embodiments of the present disclosure.

As shown in FIG. 1, an environment 100 may include an Electrical Stimulation System (ESS) 101, and a subject 103 associated with the ESS 101. The ESS 101 may be communicatively coupled with at least one User Equipment (UE) (not shown in figure) over one of, a wireless network and and a wired network. The wireless network may include, but is not limited to, Bluetooth, Bluetooth Low Energy (BLE), Wi-Fi, and Zigbee. As an example, the UE may include, but is not limited to, one of, a smartphone, a laptop, a desktop, a smartwatch, a smart television, a smart speaker, a tablet, Personal Digital Assistance (PDA), and remote monitoring device. The UE may be operated by the subject 103, a clinician, a medical staff member associated with the subject 103, and the like. The ESS 101 may be operated for providing controlled electrical stimulation 113 to a pelvic region of the subject 103, in one of, indoor environment, outdoor environment, during transition from one to another, and the like. The present description states controlling the electrical stimulation to a pelvic region of the subject. However, the method steps of controlling the electrical stimulation are applicable to other regions of the subject such as upper thigh, lower back, arms, and the like of the subject 103.

In an embodiment, the ESS 101 may receive one or more inputs 105 for initiating a session. The ESS 101 may receive the input 105 from at least one of, the UE and a User Interface (UI) of the ESS 101. The one or more inputs 105 may comprise at least one of initiating and stopping a session, and selection of one or more operating modes. Moreover, the said input 105 may be received through an analog interface or digital interface, or any combination thereof. The one or more operating modes may comprise a stimulation mode and an exercise mode.

In an embodiment, the ESS 101 may receive at least one of, an activity level 107 of a plurality of muscles in and around the pelvic region, an orientation 109 of one or more of the plurality of muscles, and a body posture 111 of the subject 103 throughout the session, upon receiving the one or more inputs 105. The plurality of muscles may comprise at least one of pelvic floor muscles and abdominal muscles of the subject 103. In an embodiment, the ESS 101 may receive at least one of, the activity level of the plurality of muscles, the orientation of one or more of the plurality of muscles, and the body posture of the subject from a determination unit associated with the ESS. The determination unit is configured to determine at least one of, the activity level of the plurality of muscles, the orientation of one or more of the plurality of muscles, and the body posture of the subject. In another embodiment, the ESS 101 may receive at least one of, the activity level of the plurality of muscles, the orientation of one or more of the plurality of muscles, and the body posture of the subject from a determination unit associated with the ESS from a machine learning model. The ESS 101 may determine whether the one or more inputs 105 pertains to a stimulation mode. The ESS 101 may monitor Electromyography (EMG) signals associated with the plurality of muscles by one or more first electrodes 229, upon determining that the one or more inputs 105 pertains to the stimulation mode. Each of the one or more first electrodes 229 may be an EMG electrode, and may be placed in and around at least one of the pelvic, the perineum, the upper thigh, the sacrum, the coccyx, and the abdominal region of the subject 103. Further, the ESS 101 may monitor the orientation 109 of one or more of the plurality of muscles and/or the body posture 111 of the subject 103 by one or more orientation sensors 233. The determination of the orientation 109 of one or more of the plurality of muscles or the body posture 111 of the subject 103 is mandatory to control the electrical stimulation to the pelvic region. Optionally, the activity level of the plurality of muscles may be determined.

Alternatively, the ESS 101 may determine that the one or more inputs 105 pertains to the exercise mode. Further, the ESS 101 may monitor the EMG signals associated with the plurality of muscles of the subject 103 by the one or more first electrodes 229 while the subject 103 is performing exercises involving the plurality of muscles in and around the pelvic region. An abdominal region of the subject is referred as a region around the pelvic region in the present description. The ESS 101 may determine the activity level 107 of the plurality of muscles based on the EMG signals. The ESS 101 may generate a biofeedback indicating contraction of the plurality of muscles based on the activity level 107. Thereafter, the ESS 101 may provide the biofeedback to the UE.

The one or more first electrodes 229 may be configured to acquire the EMG signals associated with the pelvic floor muscles as well as the abdomen muscles for measuring their respective activity levels 107. Here, the activity level 107 of the pelvic floor muscles may indicate the effect of applied electrical stimulation therapy. Whereas, the activity level 107 of the abdomen muscles may indicate presence of abdominal pressure, which may be spontaneously generated in the subject's body due to various phenomena such as coughing, laughing, etc. The effect that this may have on the activity level of the pelvic floor muscles of the subject 103, may be different from that generated by the electrical stimulation 113. Accordingly, the ESS 101 may consider such an effect as an artefact in the activity level 107 of the pelvic floor muscles and may suitably compensate for it in the output before analysis. The obtained output, which may henceforth, interchangeably, be referred to as “biofeedback”, may be measured using at least two electrodes, at least one of which may or may not act as a reference point. The biofeedback may be useful to observe and record response of the plurality of muscles, to specific set of stimulation parameters being used for the treatment.

The biofeedback may be useful to make a judgement about short term and long-term effectiveness of the treatment for the subject 103. This data may further be useful for changing the course of treatment, if required. Also, the biofeedback may be recorded and shared with the clinician associated with the subject 103 for consultation and/or further advice. The biofeedback may also be useful to detect any form of resistance which may be presented by the plurality of muscles, during a electrical stimulation treatment session.

Further, the biofeedback may be useful to detect fatigue presented by the plurality of muscles, during the electrical stimulation treatment session. Such resistances may be detected against a threshold output value of the muscle activity. The threshold output value may be arrived at by the clinician or other expert, based on experience and/or knowledge. It may be predefined into the means of programming or instructions. Moreover, a method and an associated algorithm for determination of such a threshold value may be included into the means of programming or instructions. In an embodiment, the detection may be analysed along with the detected change in both the body posture 111 and the orientation 109 of one or more of the plurality of muscles, before responding appropriately. In response to the detection of such resistance by the plurality of muscles, the ESS 101 may terminate providing the electrical stimulation 113 to ensure safety of the subject 103.

Further, the biofeedback may be useful to detect when muscle contraction is of sufficient level at a given electrical current intensity, during the electrical stimulation treatment session. Such output may be detected in the beginning of the session, as the signal intensity is slowly being increased from zero to the predefined value. In response to the detection of such output from the plurality of muscles, the ESS 101 may ensure that the intensity of the electrical current is automatically adjusted.

Further, the biofeedback may provide data which may undergo signal conditioning, for instance, through filtering before being transmitted to the UE for further analysis. Such conditioning may remove unwanted influences of the one or more orientation sensors 233, one or more second electrodes 231 on the one or more first electrodes 229, and unwanted influences of external effects such as motion artefacts, etc. Such conditioning may be useful for removal of artefact errors, etc., which may have showed up in the output, for instance, due to other body movements.

In another embodiment, the biofeedback may be provided using electronic sensor modules, which may include, but are not limited to, sensors measuring muscle movement, muscle temperature and the like. The biofeedback may also be useful to receive information about degree of muscle contraction, while the subject 103 is performing the pelvic floor muscle exercises. The subject 103 may terminate applying the electrical stimulation 113, by providing one or more inputs 105 through the UE or UI of the ESS 101, when performing pelvic floor exercises. Such operating mode may be referred to as the exercise mode.

In an embodiment, the ESS 101 may control the electrical stimulation 113 to the pelvic region based on at least one of, the activity level 107 of the plurality of muscles, the orientation 109 of one or more of the plurality of muscles, and the body posture 111 of the subject 103. The ESS 101 may trigger a pulse generator unit 235 upon determining that the one or more inputs 105 pertains to the stimulation mode. The ESS 101 may control the pulse generator unit 235 to generate a single channel of an electric current or multiple channels of electrical current with electrical stimulation parameters based on at least one of, the activity level 107 of the plurality of muscles, the orientation 109 of one or more of the plurality of muscles and the body posture 11l of the subject 103. The ESS 101 may control the pulse generator unit 235 until the activity level 107 of the plurality of muscles attain a clinically predefined value. The ESS 101 may provide the electrical stimulation 113 to the pelvic region based on the electrical current through the one or more second electrodes 231 configured in the ESS 101. Each of the one or more second electrodes 231 may be a stimulation electrode and may be placed in and around at least one of the pelvic, the perineum, the coccyx, the sacrum, the upper thigh, and the abdominal region of the subject 103.

For a particular treatment session, the ESS 101 may control one or more waveform characteristics associated with the electrical current. As an example, the one or more waveform characteristics may include, but is not limited to, a pulse geometry type, a pulse width, a frequency of the electrical current, an envelope duration, an envelope duty cycle, and signal intensity. In between multiple treatment sessions, the subject 103 may control various input parameters such as an interval time between consecutive sessions, a maximum number of sessions per day, a setting time for the session to begin automatically, and alarms for setting the session.

In an embodiment, the ESS 101 may receive a current intensity value from one of, the UE associated with the ESS 101 and the UI of the ESS 101. Further, the ESS 101 may control the pulse generator unit 235 to generate the electrical current based on the current intensity value. The ESS 101 may provide the electrical stimulation 113 to the pelvic region based on the electrical current through the one or more second electrodes 231. In some embodiments, in the ESS 101, one or more electrical parameters may be pre-set as per clinically acceptable range of values and cannot be modified by the subject 103. Such a provision may ensure that a lay person is not able to mistakenly use any value for a given parameter outside the clinically safe range.

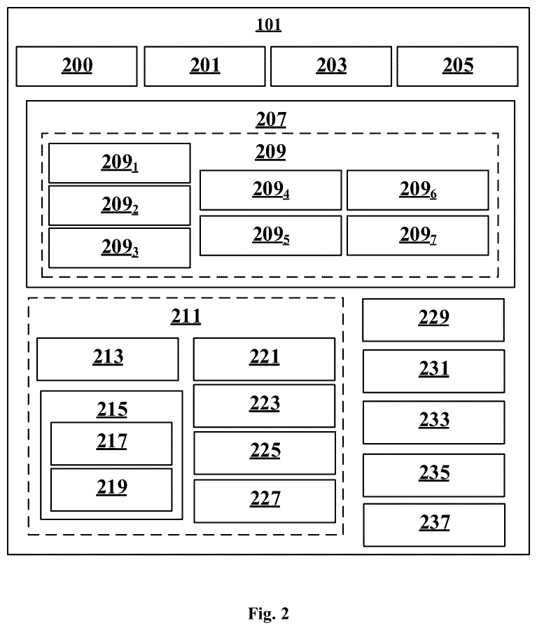

FIG. 2 shows a block diagram of an Electrical Stimulation System (ESS) in accordance with some embodiments of the present disclosure.

In some implementations, the ESS 101 may include a battery 200, an Input/Output (I/O) interface 201, a processor 203, a transceiver antenna 205, a memory 207, one or more first electrodes 229, one or more second electrodes 231, one or more orientation sensors 233, a pulse generator unit 235 and an isolation circuit 237. The battery 200 may provide power supply to components of the ESS 101. As an example, the battery 200 may be a 2000-mAh lithium rechargeable battery. Operation of the components in the ESS 101 may continue during charging of the battery 200 from AC mains electricity by a battery charger. Preferably, the components in the ESS 101 may be operated after the battery 200 is charged to prevent electrical hazards. In the ESS 101, the I/O interface 201 may receive one or more inputs 105 for initiating a session. The I/O interface 201 may also receive a current intensity value for dynamically controlling the electrical stimulation 113. Here, the I/O interface 201 may be associated with the at least one transceiver antenna 205 for performing communication with a User Equipment (UE) communicatively coupled with the ESS 101. In an embodiment, the ESS 101 may be operated with direct power supply from the AC mains, as an alternative to the battery 200.

Further, the processor 203 may receive the one or more inputs 105 for initiating the session through the transceiver antenna 205. Upon receiving the one or more inputs 105, the processor 203 may receive at least one of, an activity level 107 of a plurality of muscles in and around the pelvic region, an orientation 109 of one or more of the plurality of muscles, and a body posture 111 of the subject 103 throughout the session. Based on the determination, the processor 203 may control the electrical stimulation 113 to the pelvic region of the subject 103. Additionally, the processor 203 may receive information about the current intensity value and/or other electrical stimulation parameters through the transceiver antenna 205, and control the electrical stimulation 113 to the pelvic region of the subject 103 based on the received information. To perform the aforesaid method steps, the processor 203 may utilize various modules 211 and memory 207 of the ESS 101.

In the ESS 101, the memory 207 may store data 209 received through the I/( ) interface 201, the processor 203, the modules 211, the one or more first electrodes 229, the one or more second electrodes 231, and the one or more orientation sensors 233. In one embodiment, the data 209 may include activity level data 209₁, orientation data 209₂, body posture data 209₃, electrical stimulation parameter values 209₄, electrical current data 209₅, session data 209₆, and other data 209₇. The activity level data 209₁ may include activity levels 107 of a plurality of muscles in and around the pelvic region of the subject 103 monitored by the one or more first electrodes 229 during each session. The orientation data 209₂ may include orientation of one or more of the plurality of muscles of the subject 103 monitored by the one or more orientation sensors 233 during each session. The body posture data 209₃ may include body postures of the subject 103 monitored by the one or more orientation sensors 233 during each session. The electrical stimulation parameter values 209₄ may include current intensity value, frequency, pulse duration, duty cycle, and the like, received from at least one of, a subject 103, and a clinician associated with the subject 103 via the UE, or a User Interface (UI) associated with the ESS 101. The electrical current data 209₅ may include electrical current values determined based on at least one of, the activity level 107, the orientation 109, and the body posture 111 of the subject 103 during each session. The session data 209₆ may include start time and end time associated with each session. The other data 209₇ may store data, including temporary data and temporary files generated by the processor 203, and modules 211 for performing the various functions of the ESS 101.

In some embodiments, the data 209 stored in the memory 207 may be processed by the modules 211 of the ESS 101. In an example, the modules 211 may be communicatively coupled to the processor 203 configured in the ESS 101. The modules 211 may be present outside the memory 207 as shown in FIG. 2 and implemented as separate hardware. As used herein, the term modules may refer to an Application Specific Integrated Circuit (ASIC), an electronic circuit, a processor (shared, dedicated, or group) and memory 207 that execute one or more software or firmware programs, a combinational logic circuit, and/or other suitable components that provide the described functionality.

In some embodiments, the modules 211 may include, for example, a input module 213, a receiving module, a stimulation mode module 217, an exercise mode module 219, a control module 221, a report generation module 223, a transmitting module 225, and other modules 227. The other modules 227 may be used to perform various miscellaneous functionalities of the device 107. It will be appreciated that aforementioned modules may be represented as a single module or a combination of different modules. Furthermore, a person of ordinary skill in the art will appreciate that in an implementation, the one or more modules 211 may be stored in the memory 207, without limiting the scope of the disclosure. The said modules 211 when configured with the functionality defined in the present disclosure will result in a novel hardware.

In an embodiment, the input module 213 may receive the one or more inputs 105 for initiating the session through the I/O interface 201. The one or more inputs 105 may comprise at least one of initiating and stopping the session, and selection of one or more operating modes. The one or more operating modes may comprise a stimulation mode and an exercise mode. The input module 213 may receive the one or more inputs 105 from at least one of, the UE, and the UI of the ESS 101. Further, the input module 213 may send the one or more inputs 105 to the receiving module 215 for further processing. In some embodiments, the input module 213 may also receive the electrical stimulation parameter values through the I/O interface 201. The input module 213 may send the electrical stimulation parameter values to the control module 221 for further processing.

In an embodiment, the receiving module 215 may receive throughout the session at least one of, an activity level 107 of a plurality of muscles in and around the pelvic region, an orientation 109 of one or more of the plurality of muscles, and a body posture 111 of the subject 103 upon receiving the one or more inputs 105. The plurality of muscles may comprise at least one of pelvic floor muscles and abdominal muscles of the subject 103. The receiving module 215 may comprise a stimulation mode module 217 and an exercise mode module 219. Particularly, the receiving module 215 may determine whether the one or more inputs 105 pertains to the stimulation mode or the exercise mode. The receiving module 215 may trigger the stimulation mode module 217 to perform further processing upon determining that the one or more inputs 105 pertains to the stimulation mode. Alternatively, the receiving module 215 may trigger the exercise mode module 219 to perform further processing upon determining that the one or more inputs 105 pertains to the exercise mode.

Upon triggering, the stimulation mode module 217 may receive Electromyography (EMG) signals associated with the plurality of muscles from the one or more first electrodes 229. Each of the one or more first electrodes 229 may be an Electromyography (EMG) electrode, and may be placed in and around at least one of the pelvic, the perineum, the upper thigh, the sacrum, the coccyx, and the abdominal region of the subject 103. The stimulation mode module 217 may utilize the EMG signals received from the one or more first electrodes 229 for determining the activity level 107 of the plurality of muscles of the subject 103. Simultaneously, the stimulation mode module 217 may determine the orientation 109 of one or more of the plurality of muscles and the body posture 111 of the subject 103 utilizing the one or more orientation sensors 233. Further, the stimulation mode module 217 may send the activity level 107, the orientation 109, and the body posture 111 to the control module 221 for further processing.

As an example, the stimulation mode module 217 may detect change in the body posture, and identify a new body posture 111 and muscle orientation 109 based on relative orientation of the one or more orientation sensors 233. As an example, the stimulation mode module 217 may compare orientation of an orientation sensor positioned at lower abdomen of the subject 103 with orientation of another orientation sensor positioned at front thigh region of the subject 103. The stimulation mode module 217 may compare the orientations of the orientation sensors 233 to determine whether the subject 103 is likely to be sitting or standing. When the relative orientation is close enough to that of a sitting posture, the stimulation mode module 217 may determine that anatomical arrangement of different structures in pelvic musculature is similar to that of expected arrangement in the sitting posture. Accordingly, appropriate control may be exerted by the control module 221 to control the pulse generator unit 235 to automatically modify the electrical stimulation output. In this manner, the present disclosure may facilitate treatment modifications based on the change in the body posture, which may be effective for the subject 103. The body posture 111 may include, but is not limited to, standing, sitting, supine, squatting, and lying down. Further, the stimulation mode module 217 may detect rapid changes in the relative orientations of the one or more orientation sensors 233, which may indicate that the subject 103 is in rapid motion, such as running. In such scenarios, EMG readings may be affected, and the stimulation mode module 217 may discard the received EMG signals.

Alternatively, the exercise mode module 219 may receive the EMG signals associated with the plurality of muscles of the subject 103 by the one or more first electrodes 229 upon triggering. The exercise mode module 219 may receive the EMG signals while the subject 103 is performing exercises involving the plurality of muscles in and around the pelvic region. The exercise mode module 219 may determine the activity level 107 of the plurality of muscles based on the EMG signals. The exercise mode module 219 may generate a biofeedback indicating contraction of the plurality of muscles based on the activity level 107. Further, the exercise mode module 219 may send the biofeedback to the transmitting module 225 for further processing.

In an embodiment, the control module 221 may control the electrical stimulation 113 to the pelvic region based on at least one of, the activity level 107 of the plurality of muscles, the orientation 109 of one or more of the plurality of muscles, and the body posture 111 of the subject 103. The types of electrical stimulation 113 may include, but are not limited to, interferential stimulation, electric muscle stimulation, functional electrical stimulation, transcutaneous electrical stimulation, and neuro muscular stimulation. Particularly, the control module 221 may receive the activity level 107, the orientation 109, and the body posture 111 of the subject 103 from the stimulation mode module 217. Further, the control module 221 may trigger the pulse generator unit 235 when the one or more inputs 105 pertaining to the stimulation mode is determined. The control module 221 may determine the parameter values of the electrical stimulation output, particularly the electrical current, based on at least one of, the activity level 107 of the plurality of muscles, the orientation 109 of one or more of the plurality of muscles and the body posture 111 of the subject 103. Further, the control module 221 may control the pulse generator unit 235 to generate the electrical stimulation, particularly the electrical current based on the determined parameter values. The control module 221 may control the pulse generator unit 235 to maintain the activity level of one or more of the plurality of muscles at a value. In an embodiment, the value may be determined clinically (by a clinician or medical staff at a clinical set up or otherwise). In another embodiment, the value may be based on Maximum Voluntary Contraction of the subject. After every few days (for example, fixed period of two weeks), the ESS 101 may remind the user to try to contract their muscles voluntarily. The ESS 101 may capture muscle activity during this period to determine the value. Additionally, the control module 221 may send the activity level 107, the orientation 109, the body posture 111 and the corresponding electrical current and stimulation parameter values to one of, the report generation module 223, and the transmitting module 225 for further processing. Here, the control module 221 may select one of, the report generation module 223, and the transmitting module 225 based on configuration settings and processing capability of the UE.

In another embodiment, the control module 221 may receive the value for electrical stimulation parameters, such as current intensity value from the input module 213 for dynamically adjusting the electrical stimulation 113 by the subject 103 or a clinician associated with the subject 103. The control module 221 may control the pulse generator unit 235 to generate the electrical current based on the one or more received values, irrespective of the activity level 107 of the plurality of muscles, the orientation 109 of one or more of the plurality of muscles and the body posture 111 of the subject 103. Further, the electrical stimulation 113 may be provided to the pelvic region based on the modified electrical stimulation output through the one or more second electrodes 231. In an embodiment, the report generation module 223 may receive the activity levels, the orientations, the body postures of the subject 103 and the corresponding electrical stimulation parameter values monitored throughout the session, from the control module 221. Based on the received data, the report generation module 223 may generate a report locally at the ESS 101 for each session. Further, the report generation module 223 may send the report to the transmitting module 225.

In an embodiment, the transmitting module 225 may transmit at least one of, information associated with the activity level 107 of the plurality of muscles, information associated with the orientation 109 of one or more of the plurality of muscles, information associated with the body posture 111 of the subject 103 and information associated with the electrical current monitored throughout the session to the UE. Particularly, the transmitting module 225 may receive the activity levels, the orientations, the body postures of the subject 103 and the corresponding electrical stimulation parameter values from the control module 221, and may transmit the raw data to the UE. Here, the report may be generated locally at the UE utilizing the raw data received from the transmitting module 225 of the ESS 101. This reduces computational complexity and power consumption associated data processing for report generation at the ESS 101. The UE may also display the received raw information live on the user interface display. Alternatively, it may use a different mode-visual, auditory, and the like, to provide information about the output/biofeedback. In an alternative embodiment, the transmitting module 225 may receive the report from the report generation module 223. Further, the transmitting module 225 may transmit the generated report to the UE for information and potential future use of the subject 103 or the clinician associated with the subject 103.

In the ESS 101, the one or more first electrodes 229 may acquire EMG signals associated with the plurality of muscles of the subject 103. Each of the one or more first electrodes 229 may be an EMG electrode. The EMG electrodes may be monopolar or bipolar or any combination thereof. Further, the EMG electrodes may be non-invasive electrodes or invasive electrodes or any combination thereof. The non-invasive electrodes may include, but are not limited to, dry surface electrodes, wet surface electrodes, and semi-dry surface electrodes. The invasive electrodes may include, but are not limited to, a needle electrode and a needle comprising two fine-wire electrodes. Each of the one or more first electrodes 229 may be placed in and around at least one of the pelvic, the perineum, the upper thigh, the coccyx, the sacrum, and the abdominal region of the subject 103. As an example, the one or more first electrodes 229 may be placed in abdominal region, umbilicus region, lumbar region, sacral region, coxal region, inguinal region, pubic region and perineal region to acquire EMG signals associated with the pelvic floor muscles and the abdominal muscles of the subject 103.

In the ESS 101, the one or more second electrodes 231 may provide the electrical stimulation 113 to the pelvic region based on the electrical stimulation parameter values. Each of the one or more second electrodes 231 may be a stimulation electrode. The one or more second electrodes may respond to a single or multiple channels of electrical stimulation current generated by the pulse generator unit 235, and provide stimulation accordingly. Signal conditioning may be performed on the generated electrical current prior to providing to the one or more second electrodes. The signal conditioning may include, but is not limited to, filtering and pulse shaping. Type of the electrical current may include, but is not limited to, monophasic, sinusoidal, galvanic, faradic, or Russian, albeit as a non-limiting aspect. Characteristics of the electrical current and other electrical parameters, including number of pulses being provided, and waveform being used may vary and may, in the present disclosure, include any number and form respectively, which is acceptable as per clinical requirement. Further, the one or more second electrodes 231 may be employed for providing interferential current (also referred as interferential therapy) to the pelvic region of the subject 103. As an example, one set of second electrodes, and another set of second electrodes may apply a first electrical current and a second electrical current, respectively to the pelvic region of the subject 103, such that a target electrical current may be applied to the plurality of target muscles. The target electrical current may result from the interference of the first electrical current and the second electrical current. Both sets of the second electrodes may constitute a cathode-anode pair, each of which operates at different frequencies. Further, each of the second electrodes within a set may be operated as one of, the cathode and the anode based on the electrical stimulation 113 to be applied to the subject 103.

In an embodiment, the one or more orientation sensors 233 may measure the orientation 109 of one or more of the plurality of muscles and the body posture 111 of the subject 103. As an example, the one or more orientation sensors 233 may be positioned on thoracic part of spine of the subject 103. As an example, the one or more orientation sensors 233 may utilize inertial measurement sensors and flex sensors for detecting the orientation 109 and the body posture 111. As an example, angle values may be determined based on signals acquired by the one or more orientation sensors 233, and the orientation 109 of one or more of the plurality of muscles and the body posture 111 of the subject 103 may be determined based on the angle values.

In an embodiment, the pulse generator unit 235 may generate the electrical stimulation output, particularly the electrical current, determined by the control module 221 based on at least one of, the activity level 107 of the plurality of muscles, the orientation 109 of one or more of the plurality of muscles and the body posture 111 of the subject 103. In an embodiment, the pulse generator unit 235 may comprise a pulse generator and optionally associated peripherals. The pulse generator unit 235 may be electrically coupled with the one or more second electrodes 231, through which the electrical stimulation 113 based on the stimulation parameter values, particularly the electrical current may be provided to the pelvic region of the subject 103. The pulse generator unit 235 may generate the electrical current with determined parameter values for providing the electrical stimulation 113 to the pelvic region of the subject 103 only in the stimulation mode. In other words, the pulse generator unit 235 may remain deactivated during the exercise mode. In an alternative embodiment, the pulse generator unit 235 may generate interferential current for providing the interferential therapy to the pelvic region of the subject 103. In the interferential therapy, the pulse generator unit 235 may generate the first electrical current and the second electrical current, each having a different frequency. When the first electrical current, and the second electrical current are provided to the different channels of cathode-anode pairs, the target electrical current may be generated at the target region in and around the pelvic floor.

In the ESS 101, the isolation circuit 237 may be connected in between each of the one or more second electrodes 231 and skin of the subject 103. The isolation circuit 237 may compare the electrical current value received from each of the one or more second electrodes 231 with a predefined current value. When the electrical current value exceeds the predefined current value, the isolation circuit 237 may disconnect electrical contact of the skin from the one or more second electrodes 231, to prevent electrical hazards. As an example, a suitable transformer may be used along with other electronic peripherals to form an isolation circuit.

As an example, the one or more first electrodes 229 and the one or more second electrodes 231 may be placed in the ischial tuberosity and suprapubic regions of the subject. The mode of electrical stimulation 113 may be surface type. For a baseline resistance of 500 ohms, a peak current value, and a peak voltage value may be up to 80 mA and 40V, respectively. A pulse rate associated with a pulsed type electrical current may be within 0-100 Hz. A pulse duration associated with the pulsed type electrical current may be varied between 100 to 250 microseconds. An inter-pulse rest period associated with the electrical stimulation 113 may vary between 10 milliseconds to 2 seconds.

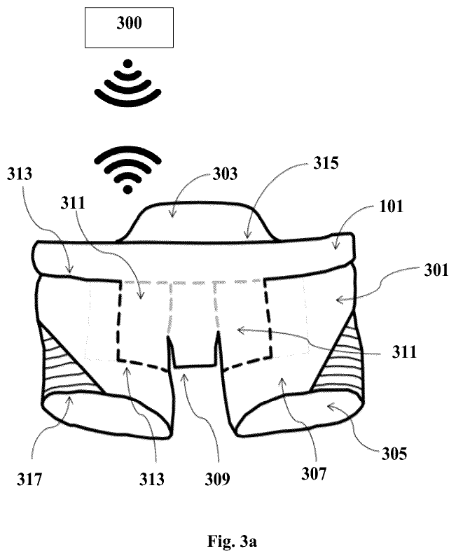

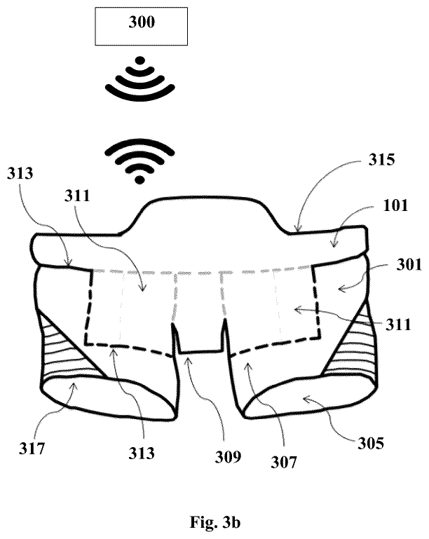

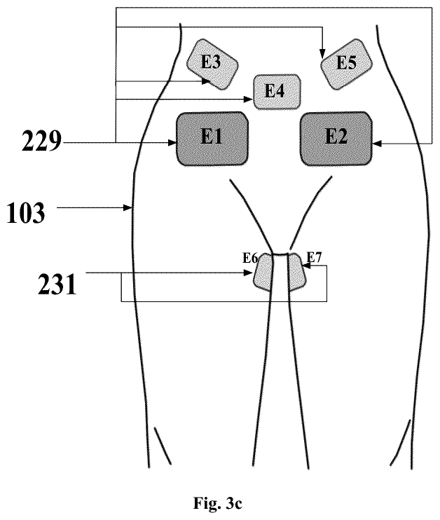

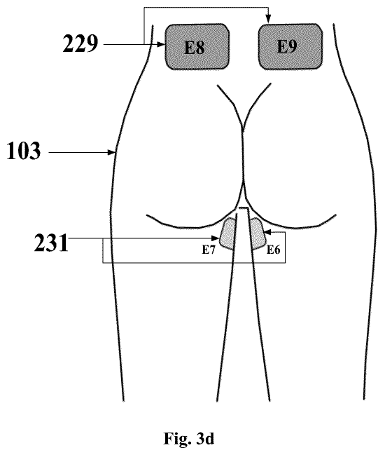

FIGS. 3a-3b show an electronic wearable unit for controlling an electrical stimulation to a pelvic region of a subject in accordance with some embodiments of the present disclosure.

FIGS. 3a, and 3b show a front view and a rear view of the electronic wearable unit 301, respectively. The electronic wearable unit 301 may comprise a non-skin contacting outer surface 307, a skin contacting inner surface 305, and an ESS 101. The non-skin contacting outer surface 307, and the skin contacting inner surface 305 may electrically insulated or non-conductive in nature. The skin contacting inner surface 305 may comprise a crotch part for removably attaching a urine collecting device 309. As an example, the urine collecting device 309 may include, but is not limited to an absorbent pad.

In the electronic wearable unit 301, the ESS 101 may be removably placed within a space bounded by the non-skin contacting outer surface 307 and the skin contacting inner surface 305. Further, the electronic wearable unit 301 may comprise one or more interlocks 313 for removably placing the ESS 101 within the space. As shown in FIGS. 3a-3b, the ESS 101 may be designed to fit inside fabric of the electronic wearable unit 301. The electronic wearable unit 301 may be shaped as shorts, such that system components such as one or more first electrodes 229, one or more orientation sensors 233, and one or more second electrodes 231 remain intact, and perform monitoring of various parameter and signals with accuracy, when the subject 103 wears the electronic wearable unit 301. The electronic wearable unit 301 comprises one or more non-electronic wearable parts and one or more electronic wearable parts. The one or more electronic wearable parts comprises at least a part of the ESS 101.

In the electronic wearable unit 301, the inner surface 305 may include one or more regions of cut outs to provide a contact interface between skin tissue of the subject 103 and the one or more second electrodes 231 for providing electrical stimulation 113. Other non-cut out regions of the inner surface 305 may be electrically insulated or non-conductive in nature. Aforesaid feature may ensure that electrical current provided through the stimulation electrodes does not come in contact with bodily fluids of the subject 103 to prevent potential hazards such as electric shock. The aforesaid feature may enable utilizing the urine collecting device 309 for managing urinary incontinence conditions of the subject 103. Insulation feature in the electronic wearable unit 301 may ensure that there is no contact between the electrical current and the urine collecting device 309, which enables the subject 103 to use the urine collecting device 309 even when the electrical stimulation 113 is provided. Additionally, the electronic wearable unit 301 may have a provision for attachment of a urinary incontinence management device. As an example, the urinary incontinence management device may include urethral insert for inserting into a urethra of the subject 103 before a specific activity. The urethral insert may act as a plug to prevent leakage and may be removed before urination.

In an embodiment, the electronic wearable unit 301 may also include pouch like holders 311 for accommodating the ESS 101. The holders 311 may comprise a first end of a connector, with which a second end of the connector, attached to the ESS 101, may be connected. In other words, one of the ends of each connector in the holder may be connected with another end of the connector, attached strategically with the ESS 101, such that the ESS 101 is locked in fixed position with the electronic wearable unit 301.

As shown in FIGS. 3a-3b, the electronic wearable unit 301 may comprise the interlocks 313 in the upper region. The ESS 101 may comprise a central electronics unit 303 at a back side 315 of top region of the ESS 101 which may act as connecting peripheries for all electronic items. The central electronics unit 303 may include, but is not limited to, a means of programming, the pulse generator unit 235, an isolation circuit, a source of power, a means of data storage, a wireless interface, connecting wires, connecting attachments and an outer holder. The outer cover of the ESS 101 may be well insulated to ensure safe contact with the skin, during the electrical stimulation 113 session. The interlocks 313 (for example, zip, velcro, and the like) may hold various wires used around the ESS 101 and may connect them all to the central electronics unit 303. The placement of the central electronics unit 303 at the back side 315 of the subject 103 may ensure maximum concealing when the subject 103 is wearing lose clothing. The periphery of the ESS 101 contains a provision for interlocking. Such an interface locks the ESS 101 in place with the electronic wearable unit 301, such that the one or more second electrodes 231 of the ESS 101 may come in contact with target regions of the skin surface of the subject 103 for stimulating target muscles based on clinical requirements. Also, such an attach-detach mechanism may facilitate the subject 103 to wash the electronic wearable unit 301 on a regular basis, making it hygienic for daily long-hour use. Further, such an attach-detach mechanism may enable the user to replace the one or more first electrodes 229 and the second one or more electrodes as per clinical requirements. Such a locked fit of the ESS 101 with the electronic wearable unit 301 may ensure that the one or more first electrodes 229 and the one or more second electrodes remain fixed in position during various movements that occur when the subject 103 performs regular day-to-day activities. Further, the one or more first electrodes 229 and the one or more second electrodes may be flexible enough to accommodate the movements of the subject 103. In a preferred embodiment, use of conductive inks for printing various electronic connections and entities, on a thin, flexible film may be employed for addressing the same. Such a thin, flexible film may easily accommodate the movements of the subject 103, while operating in a desired manner.

In an embodiment, the ESS 101 may comprise a plurality of layers of printed circuit for various electronic connections, which include, but are not limited to, inter and intra electrode connections as well as those connecting the one or more orientation sensors 233. The layer of the printed circuit, which is typically made of a thin film of a non-conductive material forms the flexible base of the same. The layers of the printed circuit, which include conductive ink are applied to the base layer, to create an electrically conductive path for transmission of current. Such a thin, flexible film may be used to administer the electrical stimulation treatment in a concealed and/or discreet manner. In an embodiment, layout design may ensure that the ESS 101 may be used in a concealed manner. The surface electrodes may be placed in the electronic wearable unit 301 such that the cross section is thin and may be concealed under regular clothing. The layout design may place the pulse generator unit 235 such that it is situated at the back side 315 of the subject 103 wearing the electronic wearable unit 301. Such a positioning may ensure that the pulse generator unit 235 may remain concealed under regular non-tight-fit clothing.

In the electronic wearable unit 301, the ESS 101 may receive one or more inputs 105 for initiating a session from a UE 300 communicatively coupled with the ESS 101 or a UI of the ESS 101. The one or more inputs 105 for initiating the session may be received from the subject 103 or a clinician associated with the subject 103. The ESS 101 may determine throughout the session at least one of, an activity level 107 of a plurality of muscles in and around the pelvic region, an orientation 109 of one or more of the plurality of muscles, and a body posture 111 of the subject 103 utilizing the one or more first electrodes 229 and the one or more orientation sensors 233. Further, the ESS 101 may control the electrical stimulation 113 to the pelvic region based on at least one of, the activity level 107 of the plurality of muscles, the orientation 109 of one or more of the plurality of muscles, and the body posture 111 of the subject 103, utilizing the pulse generator unit 235 and the one or more stimulation electrodes.

In the electronic wearable unit 301, the ESS 101 may be communicatively coupled with the UE 300 over one of, a wireless network and a wired network. In a preferred embodiment, the ESS 101 may be communicatively coupled with the UE 300 over short-range wireless networks, for example Bluetooth, as shown in FIGS. 3a-3b. The UE 300 may include, but is not limited to one of, a smartphone, a laptop, a desktop, a smartwatch, a smart television, a smart speaker, a tablet, and a Personal Digital Assistance (PDA). Configuration settings of the ESS 101 may be controlled through an external Bluetooth-interfaced UE 300 having a standalone application or a web-based interface. The configuration settings of the ESS 101 may be controlled by the subject 103, or the clinician associated with the subject 103 through the UE 300. Such a wireless interfacing enables controlling the ESS 101 without adjusting, removing, or replacing components of the electronic wearable unit 301. The wireless interfacing enables the subject 103 to use the ESS 101 in a discreet manner, without worrying about any form embarrassment, especially in a public place.

As an example, the subject 103 may wear the electronic wearable unit 301 equipped with the ESS 101 at beginning of a day. The subject 103 may enable the wireless interface of the UE 300 to administer desired form of treatment when the subject 103 is performing one of, travelling by a public transport, attending a meeting at workplace, and attending a social gathering. Use of the ESS 101 in a discreet manner through the wireless interfacing of the UE 300, may enable the subject 103 to follow the treatment regime anytime, anywhere. Here, portability of the ESS 101 as well as stimulation experience of the subject 103 are improved. This may extend electrical stimulation therapy beyond clinical settings. In other words, the subject 103 may be able to follow the treatment regime while performing regular day-to-day activities. This improves long-term compliance from the subject 103, which is important, given that main drawback of existing treatment solutions, including pelvic floor exercises, physiotherapy solutions, and so on, is the lack of long-term patient compliance.

Integration of the ESS 101 with fabric in the electronic wearable unit 301 may facilitate safe use by the subject 103 throughout a day, seven days a week. Size, material, contour and weight of the electronic wearable unit 301 may be customized to provide desired comfort to the subject 103 throughout the day. Fabric material of the electronic wearable unit 301 may be selected to meet requirements of being comfortable to wear throughout the day, ability to contain leakage, ability to contain smell, ability to stay in place, comfortable when wet and ability to keep subject's skin dry. Further, the fabric material may be selected for providing a tight elastic fit to ensure that the one or more first electrodes 229 and the one or more second electrodes 231 are in full contact with the skin of the subject 103. To ensure easy removal and wearing of the electronic wearable unit 301, elastic holders 317 are provided, such that the subject 103 may pull the electronic wearable unit 301 open with the hand when required.

In this manner, the electronic wearable unit 301 equipped with the ESS 101 may enable the subject 103 to self-administer a customised form of the electrical stimulation treatment anytime, anywhere while performing regular day to day activities. Such a treatment may be performed outside of clinical settings, by any lay person, without requiring medical expertise. The electronic wearable unit 301 equipped with the ESS 101 may provide real-time feedback to the ESS 101 for optimising the treatment for the subject 103. Thus, the electronic wearable unit 301 equipped with the ESS 101 may assist the subject 103 who lacks expertise for adjusting the electrical current value based on at least one of, an activity level 107 of a plurality of muscles in and around the pelvic region, an orientation 109 of one or more of the plurality of muscles, and a body posture 111 of the subject 103.

Further, the electronic wearable unit 301 may facilitate the subject 103 to make observations and record data about entities that may not be directly related to the electrical stimulation treatment sessions. Such entities, in the case of urinary incontinence of the subject 103 may include, but are not limited to, number of absorbent pads used, amount of leakage during a day, voiding schedules and time of holding urine. Over a course of time, such records may act as a measure of improvement in strength of the pelvic floor muscles, and consequently a measure for effectiveness of the treatment. Further, the electronic wearable unit 301 may enable the subject 103 to monitor progress levels associated with strengthening of the pelvic floor muscles of the subject 103 due to the controlled electrical stimulation 113. A positive progress may encourage the subject 103 to continue treatment. Further, a negative result may indicate the subject 103 to consult with the clinician and modify course of treatment. In an embodiment, one or more notifications may be provided to the subject 103. The one or more notifications may include, but are not limited to, status messages, warning messages, alerts, alarms, indications, signs, greeting messages, suggestions, or any combination thereof. Such notifications may be delivered by at least one of, a medium of visual, tactile and auditory feedback utilizing one or more electronic devices such as display screen, vibratory feedback, speaker, or any combination thereof.

### Examples

As an example, a subject 103 may have weakened pelvic floor muscles. To perform strengthening of the pelvic floor muscles, the subject 103 may wear the electronic wearable unit 301, which is shown in FIGS. 3a-3b. The subject 103 may operate a smartphone, in which an application may be installed for availing a controlled electrical stimulation 113. The electronic wearable unit 301 may be wirelessly coupled to the smartphone via Bluetooth. A touch input 105 for stimulation mode may be provided by the subject 103 on a touch screen of the smartphone when the subject 103 is in a supine posture.

FIGS. 3c, and 3d show a front view and a rear view of a subject 103, respectively, illustrating automatic placement of first electrodes 229 and second electrodes 231 for controlling an electrical stimulation 113 in a closed loop manner. When the subject 103 wears the electronic wearable unit 301, the one or more EMG electrodes may be automatically placed adjacent to at least one of, the perineal region, an inner thigh region, and a lower abdomen region of the subject 103. Also, the one or more stimulation electrodes may be automatically placed adjacent to at least one of, a perineal region of the subject 103 and a region extending from a sacrum to buttocks through coccyx of the subject 103, upon wearing the electronic wearable unit 301. The one or more EMG electrodes and the one or more stimulation electrodes may remain in contact with the skin of the subject 103 to measure accurately the activity level 107 of the plurality of muscles, and provide appropriate amount of electrical stimulation 113 to the target region of the subject 103.

As illustrated in FIGS. 3c-3d, seven patch EMG electrodes such as E1, E2, E3, E4, E5, E8, E9, may be automatically placed adjacent to pelvic region of the subject 103 for acquiring EMG signals associated with the plurality of muscles in and around the pelvic region of the subject 103. Two patch EMG electrodes such as E1 and E2 may be automatically placed in inguinal region (also referred as groin area) of the subject 103 as illustrated in FIG. 3e for acquiring EMG signals associated with the pelvic floor muscles of the subject 103. Further, three patch EMG electrodes such as E3, E4, E5 may be automatically placed in lower abdominal region of the subject 103 as illustrated in FIG. 3c for acquiring EMG signals associated with the abdominal muscles of the subject 103. Two patch EMG electrodes such as E8, E9 may be automatically placed in iliac region of the subject 103 as illustrated in FIG. 3d for acquiring EMG signals associated with the pelvic floor muscles of the subject 103. Also, two patch stimulation electrodes such as E6, E7 may be automatically placed in perineal region of the subject 103 as illustrated in FIGS. 3c-3d for providing the electrical stimulation 113 to the pelvic floor muscles, and nerves in and around the pelvic region.

Upon receiving the one or more inputs 105 for stimulation mode via Bluetooth from the smartphone, the ESS 101 may monitor the EMG signals through the seven patch EMG electrodes E1, E2, E3, E4, E5, E8, E9 to determine the activity level 107 of the pelvic floor muscles and the abdominal muscles of the subject 103. The ESS 101 may further monitor the muscle orientation 109 and the body posture 111 of the subject 103 by the orientation sensors 233. Based on the activity level 107, muscle orientation 109 and the body posture 111, the ESS 101 may control the pulse generator unit 235 to generate a electrical current having 100 microseconds of pulse duration, and 10 milliseconds of inter-pulse rest period. The ESS 101 may provide the electrical stimulation 113 at 50 Hz. When the subject 103 is in supine posture, the ESS 101 may maintain the electrical current at 13 milliamperes. During transition from the supine posture to a standing posture of the subject 103, the ESS 101 may gradually increase the electrical current to 29 milliamperes, such that the activity levels of the pelvic floor muscles and the abdominal muscles are maintained at the value.

In the example, due to change in the body posture 111 of the subject 103, effect of gravitational force may increase on the muscles. In the standing posture, the gravitational force may be exerted vertically downward along a direction associated with contraction and relaxation of the pelvic floor muscles. As a result, a greater amount of force may be required to induce upward movement of the pelvic floor muscles against the gravity. To induce such upward movement, the ESS 101 may increase the electrical current from 13 milliamperes to 29 milliamperes. It shall be understood that the aforesaid examples, and values thereof are for purposes of illustration only, and are not to be construed in a limiting sense.

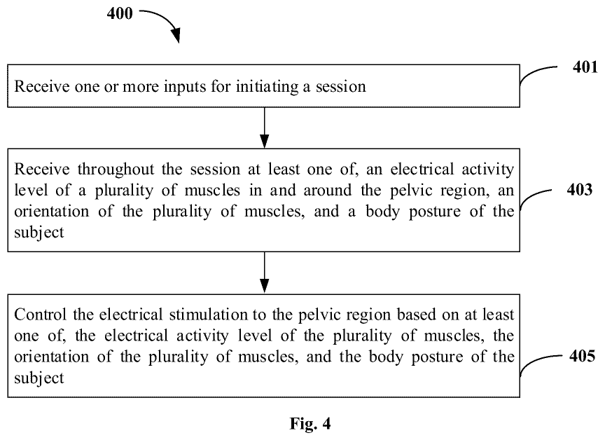

FIG. 4 shows a flow chart illustrating a method of controlling an electrical stimulation to a pelvic region of a subject in accordance with some embodiments of the present disclosure.

As illustrated in FIG. 4, the method 400 includes one or more blocks illustrating a method of controlling an electrical stimulation 113 to a pelvic region of a subject 103. The order in which the method 400 is described is not intended to be construed as a limitation, and any number of the described method blocks can be combined in any order to implement the method. Additionally, individual blocks may be deleted from the methods without departing from the scope of the subject 103 matter described herein. Furthermore, the method can be implemented in any suitable hardware, software, firmware, or combination thereof.

At block 401, the method may include receiving, by an Electrical Stimulation System (ESS) 101, one or more inputs 105 for initiating a session. The one or more inputs 105 may be received from at least one of, a User Equipment (UE) 300 communicatively coupled with the ESS 101 and a User Interface (UI) of the ESS 101. The one or more inputs 105 may comprise at least one of initiating and stopping the session, and selection of one or more operating modes. The one or more operating modes may comprise a stimulation mode and an exercise mode.

At block 403, the method may include receiving throughout the session, by the ESS, at least one of, an activity level 107 of a plurality of muscles in and around the pelvic region, an orientation 109 of one or more of the plurality of muscles, and a body posture 111 of the subject 103 upon receiving the one or more inputs 105. Here, the plurality of muscles may comprise at least one of pelvic floor muscles and abdominal muscles of the subject 103.

Particularly, it may be determined whether the one or more inputs 105 pertains to a stimulation mode. Further, Electromyography (EMG) signals associated with the plurality of muscles may be monitored by one or more first electrodes 229 to determine the activity level 107 of the plurality of muscles of the subject 103 upon determining that the one or more inputs 105 pertains to the stimulation mode. Each of the one or more first electrodes 229 may be an Electromyography (EMG) electrode, and may be placed adjacent to the pelvic region of the subject 103. Also, the orientation 109 of one or more of the plurality of muscles and the body posture 111 of the subject 103 may be monitored by one or more orientation sensors 233 upon determining that the one or more inputs 105 pertains to the stimulation mode.

Alternatively, it may be determined that the one or more inputs 105 pertains to an exercise mode. Further, the EMG signals associated with the plurality of muscles of the subject 103 may be monitored by the one or more first electrodes 229 while the subject 103 is performing exercises involving the plurality of muscles in and around the pelvic region. Based on the EMG signals, the activity level 107 of the plurality of muscles may be determined. Based on the activity level 107, a biofeedback indicating contraction of the plurality of muscles may be generated. Further, the biofeedback may be provided to the UE 300.

At block 405, the method may include controlling, by the ESS 101, the electrical stimulation 113 to the pelvic region based on at least one of, the activity level 107 of the plurality of muscles, the orientation 109 of one or more of the plurality of muscles, and the body posture 111 of the subject 103. Particularly, a pulse generator unit 235 configured in the ESS 101 may be triggered upon determining that the one or more inputs 105 pertains to the stimulation mode. Based on at least one of, the activity level 107 of the plurality of muscles, the orientation 109 of one or more of the plurality of muscles and the body posture 111 of the subject 103, the pulse generator unit 235 may be controlled to generate an electrical current. Further, the electrical stimulation 113 may be provided to the pelvic region based on the electrical current through one or more second electrodes 231 configured in the ESS 101. Each of the one or more second electrodes 231 may be a stimulation electrode and may be placed in and around at least one of the pelvic, the perineum, the coccyx, the sacrum, the upper thigh, and the abdominal region of the subject 103. Here, the pulse generator unit 235 may be controlled until the activity level 107 of the plurality of muscles attains a value.

The electrical stimulation 113 to the pelvic region may also be controlled based on one or more inputs 105 comprising an electrical stimulation parameter value. Particularly, the electrical stimulation parameter value may be received from one of, the UE 300 communicatively coupled with the ESS 101 and the UI of the ESS 101. Based on the electrical stimulation parameter values, the pulse generator unit 235 may be controlled to generate the electrical current. Based on the electrical current, the electrical stimulation 113 may be provided to the pelvic region through the one or more second electrodes 231.

Further, at least one of, information associated with the activity level 107 of the plurality of muscles, information associated with the orientation 109 of one or more of the plurality of muscles, information associated with the body posture 111 of the subject 103 and information associated with the electrical current monitored throughout the session may be transmitted to the UE 300. At the UE 300, the report may be generated based on the received information.

Alternatively, a report for the session comprising at least one of, information associated with the activity level 107 of the plurality of muscles, information associated with the orientation 109 of one or more of the plurality of muscles, information associated with the body posture 111 of the subject 103 and information associated with the electrical current monitored throughout the session may be generated. Further, the generated report may be transmitted to the UE 300.

The present disclosure discloses a method for rehabilitation of the pelvic region of the subject by controlling the electrical stimulation to the pelvic region. In an embodiment, the ESS may receive at least one of, the activity level of the plurality of muscles in and around the pelvic region, the orientation of one or more of the plurality of muscles, and the body posture of the subject for rehabilitation of the pelvic region.

The present disclosure also provides a method of managing and/or treating disorder associated with pelvic region of a subject, said method comprising rehabilitation of the pelvic region by controlling an electrical stimulation to the pelvic region.

In some embodiments, the electrical stimulation to the pelvic region is controlled by the method as described above, that comprises receiving, by an Electrical Stimulation System (ESS), one or more inputs for initiating a session; receiving throughout the session, by the ESS, at least one of, an activity level of a plurality of muscles in and around the pelvic region, an orientation of one or more of the plurality of muscles, and a body posture of the subject; and controlling, by the ESS, the electrical stimulation to the pelvic region based on at least one of, the activity level of the plurality of muscles, the orientation of one or more of the plurality of muscles, and the body posture of the subject.

In some embodiments, the disorder is selected from a group comprising conditions related to bladder, weak pelvic floor muscles, weak sphincter muscles, nerve injury, requirement of sacral nerve, pudendal nerve, avulsion of a muscle, rupture of connective tissue under the skin of pelvic floor and one or more conditions related to pelvic floor dysfunction comprising, urinary incontinence, anal and fecal incontinence, urinary frequency, weak vaginal muscle tone, pelvic surgeries, pelvic organ prolapse, and sexual dysfunction, or any combination thereof.

The present disclosure therefore also relates to a method of managing and/or treating for example, urinary incontinence in a subject, said method comprising rehabilitation of the pelvic region by controlling an electrical stimulation to the pelvic region as described above.

Accordingly, in some embodiments, the urinary incontinence is managed and/or treated by controlling the electrical stimulation to the pelvic region of the subject, by a method that comprises: receiving, by an Electrical Stimulation System (ESS), one or more inputs for initiating a session; receiving throughout the session, by the ESS, at least one of, an activity level of a plurality of muscles in and around the pelvic region, an orientation of one or more of the plurality of muscles, and a body posture of the subject; and controlling, by the ESS, the electrical stimulation to the pelvic region based on at least one of, the activity level of the plurality of muscles, the orientation of one or more of the plurality of muscles, and the body posture of the subject.

### Computer System

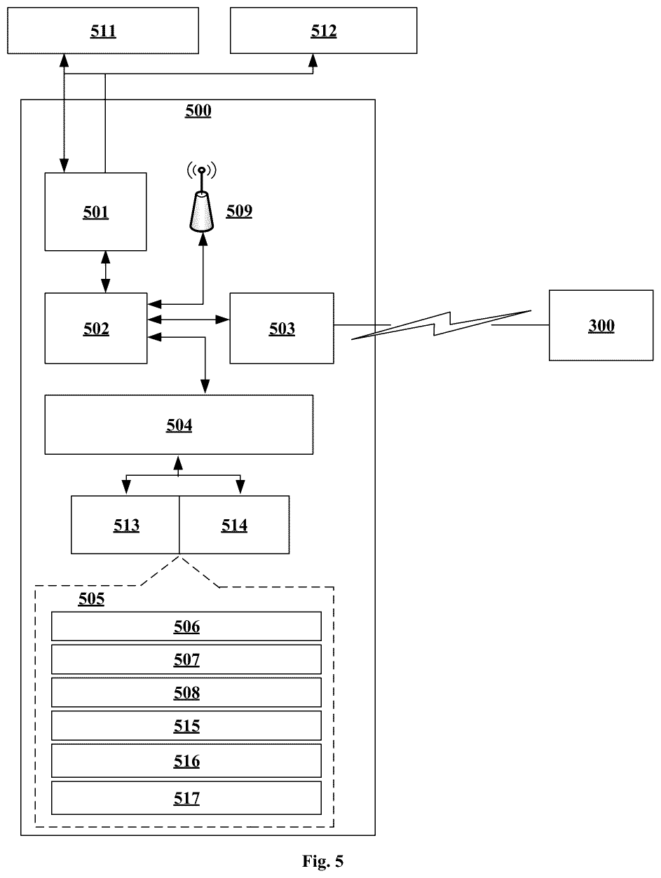

FIG. 5 illustrates a block diagram of an exemplary computer system 500 for implementing embodiments consistent with the present disclosure. In an embodiment, the computer system 500 may be a system for synchronizing a first transceiver device 101 and one or more second transceiver devices 105 for Wi-Fi sharing. The computer system 500 may include a central processing unit (“CPU” or “processor”) 502. The processor 502 may comprise at least one data processor for executing program components for executing user or system-generated business processes. The processor 502 may include specialized processing units such as integrated system (bus) controllers, memory management control units, floating point units, graphics processing units, digital signal processing units, etc.

The processor 502 may be disposed in communication with one or more input/output (I/O) devices (511 and 512) via I/O interface 501. The I/O interface 501 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc. Using the I/O interface 501, the computer system 500 may communicate with one or more I/O devices 511 and 512.

In some embodiments, the processor 502 may be disposed in communication with a wireless communication network via a network interface 503. The network interface 503 may communicate with the wireless communication network. The network interface 503 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The wireless communication network can be implemented as one of the several types of networks, such as intranet or Local Area Network (LAN) and such within the organization. The wireless communication network may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 201 may include a variety of network devices, including routers, bridges, servers, computing devices, storage devices, etc.

In some embodiments, the processor 502 may be disposed in communication with a memory 505 (e.g., RAM 513, ROM 514, etc. as shown in FIG. 5) via a storage interface 504. The storage interface 504 may connect to memory 505 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 505 may store a collection of program or database components, including, without limitation, user/application 506, an operating system 507, a web browser 508, mail client 515, mail server 516, web server 517 and the like. In some embodiments, computer system 500 may store user/application data 506, such as the data, variables, records, etc. as described in this invention. Such databases may be implemented as fault-tolerant, relational, scalable, secure databases such as Oracle® or Sybase®.

The operating system 507 may facilitate resource management and operation of the computer system 500. Examples of operating systems include, without limitation, APPLE MACINTOSH® OS X, UNIX®, UNIX-like system distributions (E.G., BERKELEY SOFTWARE DISTRIBUTION™ (BSD), FREEBSD™, NETBSD™, OPENBSD™, etc.), LINUX DISTRIBUTIONS™ (E.G., RED HAT™, UBUNTU™, KUBUNTU™, etc.), IBM™ OS/2, MICROSOFT™ WINDOWS™ (XP™, VISTA™/7/8, 10 etc.), APPLE® IOS™, GOOGLE® ANDROID™, BLACKBERRY® OS, or the like. A user interface may facilitate display, execution, interaction, manipulation, or operation of program components through textual or graphical facilities. For example, user interfaces may provide computer interaction interface elements on a display system operatively connected to the computer system 500, such as cursors, icons, check boxes, menus, windows, widgets, etc. Graphical User Interfaces (GUIs) may be employed, including, without limitation, APPLE MACINTOSH® operating systems, IBM™ OS/2, MICROSOFT™ WINDOWS™ (XP™, VISTA™/7/8, 10 etc.), Unix® X-Windows, web interface libraries (e.g., AJAX™, DHTML™, ADOBE® FLASH™, JAVASCRIPT™, JAVA™, etc.), or the like.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

### Advantages of the Embodiment of the Present Disclosure are Illustrated Herein

In an embodiment, the present disclosure provides a method, an Electrical Stimulation System (ESS), and an electronic wearable unit for controlling an electrical stimulation to a pelvic region of a subject.

In an embodiment, the present disclosure facilitates dynamically controlling the electrical stimulation to the pelvic region of the subject based on at least one of, an activity level of plurality of muscles in and around the pelvic region, an orientation of one or more of the plurality of muscles, and a body posture of the subject. This enables the subject to administer stimulation-based treatment in a portable manner, and further extend it to ambulatory use in multiple body postures without interrupting regular day-to-day activities.

In an embodiment, the present disclosure ensures accuracy in adjusting stimulation parameters for effective treatment. Here, the electrical stimulation is automatically adjusted based on the real-time activity level of the plurality of muscles, which is determined from real-time EMG signals associated with the plurality of muscles acquired by one or more EMG electrodes. As opposed to open loop control mechanism provided in the conventional systems, the present disclosure provides a closed loop control mechanism, in which the electrical stimulation is controlled based on real-time feedback, thereby ensuring accuracy. Further, effect of gravity on the pelvic floor muscles is automatically considered while controlling the electrical stimulation due to the real-time feedback. This also enables customising the stimulation-based treatment specific to the subject, thereby improving efficacy.

In an embodiment, the present disclosure facilitates controlling the electrical stimulation based on operating modes. In the stimulation mode, the real-time feedback based on EMG readings acts as a response for the closed loop system for accurately controlling the electrical stimulation. Whereas in the exercise mode, the real-time feedback based on EMG readings acts as biofeedback for indicating the subject regarding contraction of the plurality of muscles while the subject is performing exercises involving the plurality of muscles.

In an embodiment, the present disclosure provides an electronic wearable unit which enables the subject to control the electrical stimulation and manage condition of urinary incontinence simultaneously. In the electronic wearable unit, a skin contacting inner surface comprising a crotch part is provided for removably attaching a urine collecting device. This enables the subject to attach and detach the urine collecting device without removing the ESS. The method and/or system of the present disclosure is intended for use as a medical treatment for rehabilitation of pelvic floor muscles.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise. The enumerated listing of items does not imply that any or all the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device or article is described herein (whether they cooperate), it will be clear that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed heroin are for purposes of illustration and are not intended to be limiting, with the true scope and spirit being indicated by the following claims.

Referral Numerals:

| Reference Number | Description |
|---|---|
| 100 | Environment |
| 101 | Electrical Stimulation System (ESS) |
| 103 | Subject |
| 105 | One or more inputs |
| 107 | Activity level |
| 109 | Orientation |
| 111 | Body posture |
| 113 | Electrical stimulation |
| 200 | Power Supply |
| 201 | I/O interface |
| 203 | Processor |
| 205 | Transceiver antenna |
| 207 | Memory |
| 209 | Data |
| 209₁ | Activity level data |
| 209₂ | Orientation data |
| 209₃ | Body posture data |
| 209₄ | Electrical stimulation parameter values |
| 209₅ | Electrical current data |
| 209₆ | Session data |
| 209₇ | Other data |
| 211 | Modules |
| 213 | Input module |
| 215 | Receiving module |
| 217 | Stimulation mode module |
| 219 | Exercise mode module |
| 221 | Control module |
| 223 | Report generation module |
| 225 | Transmitting module |
| 227 | Other modules |
| 229 | First electrodes |
| 231 | Second electrodes |
| 233 | Orientation sensors |
| 235 | Pulse generator unit |
| 237 | Isolation circuit |
| 300 | User Equipment (UE) |
| 301 | Electronic wearable unit |
| 303 | Central electronics unit |
| 305 | Skin contacting inner surface |
| 307 | Non-skin contacting outer surface |
| 309 | Urine collecting device |
| 311 | Holders |
| 313 | Interlocks |
| 315 | Back side |
| 317 | Elastic holders |
| 500 | Computer system |
| 501 | I/O Interface |
| 502 | Processor |
| 503 | Network interface |
| 504 | Storage interface |
| 505 | Memory |
| 506 | User/Application |
| 507 | Operating system |
| 508 | Web browser |
| 509 | Transceiver |
| 511 | Input device |
| 512 | Output device |
| 513 | RAM |
| 514 | ROM |
| 515 | Mail client |
| 516 | Mail server |
| 517 | Web server |

We claim:

1. A method of controlling an electrical stimulation to a pelvic region of a subject, the method comprising of:
receiving, by an Electrical Stimulation System (ESS), one or more inputs for initiating a session;
receiving throughout the session, by the ESS, at least one of, an activity level of a plurality of muscles in and around the pelvic region, an orientation of one or more of the plurality of muscles, and a body posture of the subject; and
controlling, by the ESS, the electrical stimulation to the pelvic region based on at least one of, the activity level of the plurality of muscles, the orientation of one or more of the plurality of muscles, and the body posture of the subject.

2. The method as claimed in claim 1, wherein the one or more inputs are received from at least one of, a User Equipment (UE) communicatively coupled with the ESS and a User Interface (UI) of the ESS.

3. The method as claimed in claim 1, wherein the one or more inputs comprises at least one of, initiating the session and selection of one or more operating modes.

4. The method as claimed in claim 3, wherein the one or more operating modes comprise a stimulation mode and an exercise mode.

5. The method as claimed in claim 1, wherein the plurality of muscles comprises at least one of, pelvic floor muscles and abdominal muscles of the subject.

6. The method as claimed in claim 1, wherein the receiving comprises at least one of,
receiving at least one of, the activity level of the plurality of muscles, the orientation of one or more of the plurality of muscles, and the body posture of the subject from a determination unit associated with the ESS, wherein the determination unit is configured to determine at least one of, the activity level of the plurality of muscles, the orientation of one or more of the plurality of muscles, and the body posture of the subject; and
receiving at least one of, the activity level of the plurality of muscles, the orientation of one or more of the plurality of muscles, and the body posture of the subject from a determination unit associated with the ESS from a machine learning model.

7. The method as claimed in claim 6, wherein determining at least one of, the activity level of the plurality of muscles, the orientation of one or more of the plurality of muscles, and the body posture of the subject comprises:
determining whether one or more inputs pertains to a stimulation mode;
monitoring at least one of:
signals associated with the plurality of muscles by one or more first electrodes to determine the activity level of the plurality of muscles of the subject;
orientation of one or more of the plurality of muscles by one or more orientation sensors; and
the body posture of the subject by the one or more orientation sensors.

8. The method as claimed in claim 7, wherein each of the one or more first electrodes placed adjacent to the pelvic region of the subject.

9. The method as claimed in claim 1, wherein controlling the electrical stimulation to the pelvic region comprises:
triggering a pulse generator unit configured in the ESS upon determining that the one or more inputs pertains to a stimulation mode;
controlling the pulse generator unit to generate an electrical current based on at least one of, the activity level of the plurality of muscles, the orientation of one or more of the plurality of muscles and the body posture of the subject; and
providing the electrical stimulation to the pelvic region based on the electrical current through one or more second electrodes configured in the ESS.

10. The method as claimed in claim 9, wherein the pulse generator unit is controlled to maintain the activity level of one or more of the plurality of muscles at a value.

11. The method as claimed in claim 9, wherein each of the one or more second electrodes is a stimulation electrode and is placed in and around at least one of the pelvic, the perineum, the coccyx, the sacrum, the upper thigh, and the abdominal region of the subject.

12. The method as claimed in claim 6, further comprises:
determining whether one or more inputs pertains to an exercise mode;
monitoring the signals associated with the plurality of muscles of the subject by the one or more first electrodes while the subject is performing exercises involving the plurality of muscles in and around the pelvic region;
determining the activity level of the plurality of muscles based on the signals;
generating a biofeedback indicating contraction of the plurality of muscles based on the activity level; and
providing the biofeedback to the UE.

13. The method as claimed in claim 1, further comprises:
receiving, by the ESS, one or more electrical stimulation parameter values from one of, a User Equipment (UE) communicatively coupled with the ESS and a User Interface (UI) of the ESS;
controlling the pulse generator unit to generate the electrical current based on the current intensity value; and
providing the electrical stimulation to the pelvic region based on the electrical current through the one or more second electrodes.

14. The method as claimed in claim 1, further comprises:
transmitting at least one of, information associated with the activity level of the plurality of muscles, information associated with the orientation of one or more of the plurality of muscles, information associated with the body posture of the subject and information associated with the electrical current monitored throughout the session to the UE.

15. The method as claimed in claim 1, further comprises:
generating a report for the session comprising at least one of, information associated with the activity level of the plurality of muscles, information associated with the orientation of one or more of the plurality of muscles, information associated with the body posture of the subject and information associated with the electrical current monitored throughout the session; and
transmitting the generated report to the UE.

16. An Electrical Stimulation System (ESS) for controlling an electrical stimulation to a pelvic region of a subject, the ESS comprises:
a processor; and
a memory communicatively coupled to the processor, wherein the memory stores the processor-executable instructions, which, on execution, causes the processor to:
receive one or more inputs for initiating a session;
receive throughout the session at least one of, an activity level of a plurality of muscles in and around the pelvic region, an orientation of one or more of the plurality of muscles, and a body posture of the subject, and
control the electrical stimulation to the pelvic region based on at least one of, the activity level of the plurality of muscles, the orientation of one or more of the plurality of muscles, and the body posture of the subject.

17. The ESS as claimed in claim 16, wherein the one or more inputs are received from at least one of, a User Equipment (UE) communicatively coupled with the ESS and a User Interface (UI) of the ESS.

18. The ESS as claimed in claim 16, wherein the one or more inputs comprises at least one of, initiating the session and selection of one or more operating modes.

19. The ESS as claimed in claim 16, wherein the one or more operating modes comprise a stimulation mode and an exercise mode.

20. The ESS as claimed in claim 16, wherein the plurality of muscles comprises at least one of pelvic floor muscles and abdominal muscles of the subject.

21. The ESS as claimed in claim 16, wherein the processor is configured to,
receiving at least one of, the activity level of the plurality of muscles, the orientation of one or more of the plurality of muscles, and the body posture of the subject from a determination unit associated with the ESS, wherein the determination unit is configured to determine at least one of, the activity level of the plurality of muscles, the orientation of one or more of the plurality of muscles, and the body posture of the subject; and
receiving at least one of, the activity level of the plurality of muscles, the orientation of one or more of the plurality of muscles, and the body posture of the subject from a determination unit associated with the ESS from a machine learning model.

22. The ESS as claimed in claim 21, wherein the processor is configured to:
determine whether the one or more inputs pertains to a stimulation mode;
monitor at least one of:
signals associated with the plurality of muscles by one or more first electrodes to determine the activity level of the plurality of muscles of the subject;
the orientation of one or more of the plurality of muscles by one or more orientation sensors; and
the body posture of the subject by the one or more orientation sensors.

23. The ESS as claimed in claim 22, wherein each of the one or more first electrodes is placed adjacent to the pelvic region of the subject.

24. The ESS as claimed in claim 16, wherein the processor is configured to:
trigger a pulse generator configured in the ESS upon determining that the one or more inputs pertains to the stimulation mode;
control the pulse generator to generate an electrical current based on at least one of, the activity level of the plurality of muscles, the orientation of one or more of the plurality of muscles and the body posture of the subject; and
provide the electrical stimulation to the pelvic region of the subject based on the electrical current through one or more second electrodes configured in the ESS.

25. The ESS as claimed in claim 24, wherein the processor controls the pulse generator unit maintain the activity level of one or more of the plurality of muscles at a value.

26. The ESS as claimed in claim 24, wherein each of the one or more second electrodes is a stimulation electrode, and is placed adjacent to the pelvic region of the subject.

27. The ESS as claimed in claim 16, wherein the processor is configured to:
determine whether the one or more inputs pertains to an exercise mode;
monitor the signals associated with the plurality of muscles of the subject by the one or more first electrodes while the subject is performing exercises involving the plurality of muscles in and around the pelvic region;
determine the activity level of the plurality of muscles based on the signals;
generate a biofeedback indicating contraction of the plurality of muscles based on the activity level; and
provide the biofeedback to the UE.

28. The ESS as claimed in claim 16, wherein the processor is configured:
receive one or more electrical stimulation parameter values from one of, a User Equipment (UE) communicatively coupled with the ESS and a User Interface (UI) of the ESS;
control the pulse generator to generate the electrical current based on the current intensity value; and
provide the electrical stimulation to the pelvic region based on the electrical current through the one or more second electrodes.

29. The ESS as claimed in claim 16, wherein the processor is configured to:
transmit at least one of, information associated with the activity level of the plurality of muscles, information associated with the orientation of one or more of the plurality of muscles, information associated with the body posture of the subject and information associated with the electrical current monitored throughout the session to the UE.

30. The ESS as claimed in claim 16, wherein the processor is configured to:
generate a report for the session comprising at least one of, information associated with the activity level of the plurality of muscles, information associated with the orientation of one or more of the plurality of muscles, information associated with the body posture of the subject and information associated with the electrical current monitored throughout the session; and
transmit the generated report to the UE.

31. An electronic wearable unit for controlling an electrical stimulation to a pelvic region of a subject, the electronic wearable unit comprises:
one or more non-electronic wearable parts; and
one or more electronic wearable parts comprising at least a part of an Electrical Stimulation System (ESS), wherein the ESS is configured to:
receive one or more inputs for initiating a session;
receive throughout the session at least one of, an activity level of a plurality of muscles in and around the pelvic region, an orientation of one or more of the plurality of muscles, and a body posture of the subject; and
control the electrical stimulation to the pelvic region based on at least one of, the activity level of the plurality of muscles, the orientation of one or more of the plurality of muscles, and the body posture of the subject.

32. The electronic wearable unit as claimed in claim 31, further comprises one or more interlocks for removably attaching the electronic wearable part from the non-electronic wearable part.

33. The electronic wearable unit as claimed in claim 31, further comprises a crotch part for removably attaching a urine collecting device.

34. A method for rehabilitation of pelvic region of a subject by controlling an electrical stimulation to the pelvic region, the method comprising of:
receiving, by an Electrical Stimulation System (ESS), one or more inputs for initiating a session;
receiving throughout the session, by the ESS, at least one of, an activity level of a plurality of muscles in and around the pelvic region, an orientation of one or more of the plurality of muscles, and a body posture of the subject; and
controlling, by the ESS, the electrical stimulation to the pelvic region based on at least one of, the activity level of the plurality of muscles, the orientation of one or more of the plurality of muscles, and the body posture of the subject.

35. A method of managing disorder associated with pelvic region of a subject, said method comprising rehabilitation of the pelvic region by controlling an electrical stimulation to the pelvic region by method as claimed in claim 1.

36. The method as claimed in claim 35, wherein the disorder is selected from a group comprising conditions related to bladder, weak pelvic floor muscles, weak sphincter muscles, nerve injury, requirement of sacral nerve, pudendal nerve, avulsion of a muscle, rupture of connective tissue under the skin of pelvic floor and one or more conditions related to pelvic floor dysfunction comprising, urinary incontinence, anal and fecal incontinence, urinary frequency, weak vaginal muscle tone, pelvic surgeries, pelvic organ prolapse, and sexual dysfunction, or any combination thereof.
